(19) United States
(12) Patent Application Publication
(10) Pub. No.: US 2010/0297235 A1
(43) Pub. Date: Nov. 25, 2010

(54) Vascular puncture closure systems, devices, and methods using biocompatible synthetic hydrogel compositions

(75) Inventor: Olexander Hnojewyj, Emerald Hills, CA (US)

Correspondence Address: RYAN KROMHOLZ & MANION, S.C., POST OFFICE BOX 26618, MILWAUKEE, WI, 53226, US

(73) Assignee: CPC of America, Inc.

(21) Appl. No.: 12/454,593

(22) Filed: May 20, 2009

(51) Int. Cl. A61K 31/765; A61P 17/02; A61P 7/00; A61B 17/03

(52) U.S. Cl. 424/486; 424/78.37; 606/214

(57) ABSTRACT

A hydrogel composition for application to vascular puncture site of an animal to arrest bleeding and promote hemostasis mixes a biocompatible, synthetic, electrophilic polymer component comprising a poly(ethylene glycol) (PEG) Succinimidyl Glutarate having a functionality of four and a molecular weight of about 10,000 g/mole, with a biocompatible, synthetic, nucleophilic polymer component comprising a blend of a poly(ethylene glycol) (PEG) Amine having a functionality of four and a molecular weight of about 10,000 g/mole, and a Poly-L-Lysine hydrobromide having a molecular weight of greater than about 8000 g/mole.

### FIELD OF INVENTION

The invention relates to systems, devices, methods, and compositions for achieving hemostasis at a vascular puncture site formed, e.g., as part of an interventional, catheter-based, endovascular procedure.

### BACKGROUND OF THE INVENTION

The Seldinger technique is a well-established procedure in clinical practice used to introduce catheters, probes, electrodes, etc. into blood vessels. The Seldinger technique permits safe access to blood vessels. It is named after Dr. Sven-Ivar Seldinger, a Swedish radiologist who introduced the procedure in 1953.

In the Seldinger techniques, a targeted blood vessel is punctured with a sharp hollow needle called a trocar, with ultrasound guidance, if necessary. A guidewire is then advanced through the lumen of the trocar, and the trocar is withdrawn. A “sheath” or blunt cannula can now be passed over the guidewire into the cavity or vessel. The sheath can be used to introduce catheters or other devices to perform endoluminal (inside the hollow organ) procedures, such as angioplasty. Percutaneous catheter-based procedures, such as thermoablation, angioplasty, embolization, or biopsy, may be performed. Upon completion of the desired procedure, the sheath is withdrawn.

There are about 15 million procedures performed worldwide each year using interventional, catheter-based techniques. This number is expanding yearly as aging populations demand less invasive treatments and advances in the technologies themselves extend transcatheter therapies to a wider range of patients.

For every percutaneous procedure performed, there is a puncture site that needs to be addressed. Each vascular puncture procedure creates a hole that is made in an artery or vein to allow passage of the catheter and devices into the vasculature. These punctures are typically made in vessels associated with high-pressure blood flow (e.g., the femoral artery) and must be stabilized or they will continue to bleed. An essential part of each and every transcatheter procedure is the achievement of hemostasis at the site of the vascular puncture.

The most common way of achieving puncture site hemostasis is by applying manual pressure to the site. Manual compression is time consuming, resource intensive, and uncomfortable for patients, who must lie still with manual compression on their groin for six to eighteen hours following their procedure. Manual compression has been around since catheter-based procedures were first devised in the 1950s. Although manual compression works, it is far from ideal and to some, the manual compression methodology seems comparatively archaic.

Vascular closure devices (VCD's) were first developed in the mid-1990s to deal with concerns of high rates of access site bleeding associated with percutaneous interventional, catheter-based techniques procedures. Despite the goals of improving patient outcomes, patient comfort, and catheterization laboratory efficiency, VCD adoption has not paralleled the rapid pace of other interventional cardiology technologies. It has been estimated that only one-third of patients received a VCD. Despite the considerable knowledge that has been gained in the past 10 years regarding the strengths, weaknesses, and potential applications of VCD, concerns about costs of VCD use and lack of superiority over manual compression have dampened enthusiasm for their routine use.

### SUMMARY OF THE INVENTION

The invention provides compositions, systems, and methods for achieving hemostasis at vascular puncture sites, allowing a patient to return to ambulatory status quickly following a vascular access procedure.

One aspect of the invention comprises a biocompatible, synthetic, electrophilic (i.e., electron withdrawing) polymer component mixed, with a biocompatible, synthetic, nucleophilic (i.e., electron donating) polymer component. By “synthetic,” it is meant that the component is chemically synthesized in the laboratory or industrially or produced using recombinant DNA technology.

The synthetic polymer components, when mixed in a liquid state, react by cross-linking, forming a solid matrix composition, or hydrogel. By “cross-linking,” it is meant that the hydrogel composition contains intermolecular crosslinks and optionally intramolecular crosslinks as well, arising from the formation of covalent bonds. The term “hydrogel” or “hydrogel composition” refers to a state of matter comprising a cross-linked polymer network swollen in a liquid medium. According to this aspect of the invention, the hydrogel transforms over time by physiologic mechanisms from a solid state back to a biocompatible liquid state, which can be cleared by the body. The transformation can occur, e.g., by hydrolysis of the polymer backbone.

The electrophilic component and/or the nucleophilic component can include additive components, which can affect the physical and mechanical characteristics of the composition.

In a preferred embodiment, the biocompatible, synthetic, electrophilic polymer component comprises a poly(ethylene glycol) (PEG) Succinimidyl Glutarate having a functionality of four—or, in short hand, 4-Arm PEG Succinimidyl Glutarate (PEG-SG)—having a molecular weight of about 10,000 g/mole. In a preferred embodiment, the biocompatible, synthetic, nucleophilic polymer component comprises a blend of a poly(ethylene glycol) (PEG) Amine having a functionality of four—or, in short hand, 4-Arm PEG Amine—having a molecular weight of about 10,000 g/mole, and a Poly-L-Lysine hydrobromide having a molecular weight of greater than about 8000 g/mole.

Other features and advantages of the various aspects of the inventions are set forth in the following specification and drawings, as well as being defined in the appended claims.

### BRIEF DESCRIPTION OF THE DRAWINGS

FIG. 1 is a largely diagrammatic view of a system for creating a biocompatible and biodegradable barrier to seal a vascular puncture site that comprises a biocompatible, synthetic electrophilic polymer component that is mixed with a biocompatible, synthetic nucleophilic component to form a hydrogel matrix.

FIG. 2A is an illustrative embodiment of the system shown in FIG. 1 in kit form.

FIGS. 2B to 2F illustrate the manipulation of the constituents in the kit shown in FIG. 2A to form the electrophilic polymer component and nucleophilic polymer component prior to use.

FIG. 3A is an illustrative embodiment of the system shown in FIG. 1 in kit form.

FIGS. 3B to 3F illustrate the manipulation of the constituents in the kit shown in FIG. 2A to form the electrophilic polymer component and nucleophilic polymer component prior to use.

FIGS. 4A to 4I illustrate the manipulation of a delivery device to deliver the electrophilic polymer component and nucleophilic polymer component to create a hydrogel matrix that seals a vascular puncture site.

FIGS. 5 and 6 are graphs showing the gelation characteristics of a hydrogel matrix that embodies the features of the invention.

FIGS. 7 and 8 are graphs showing the gelation characteristics of hydrogel matrixes that do not embody the features of the invention.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention, which is defined by the claims.

A. System Overview

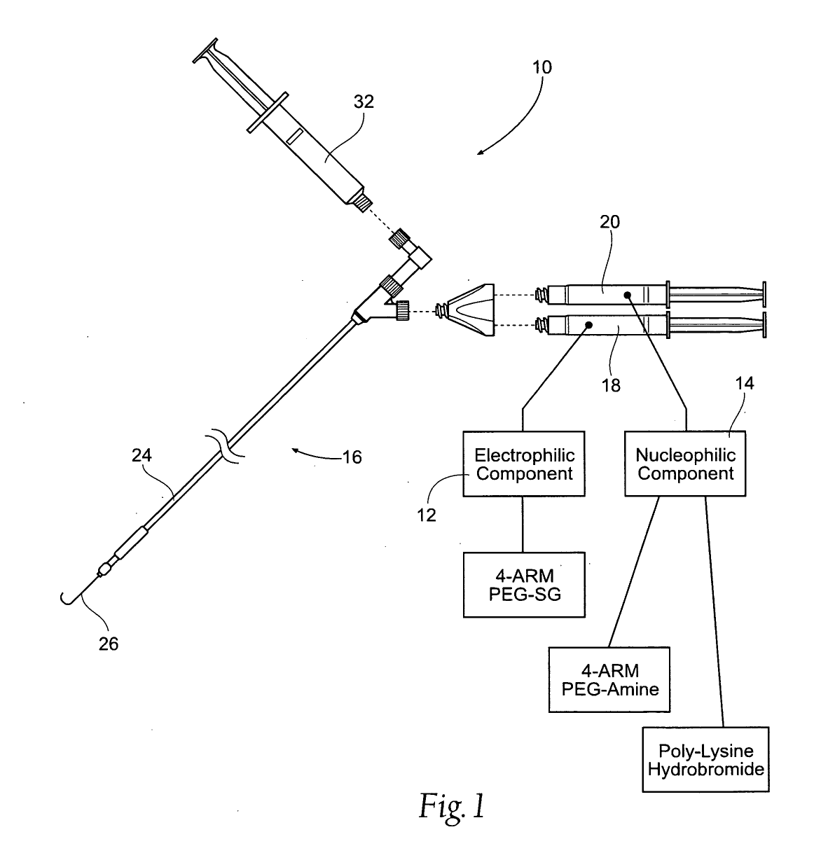

FIG. 1 shows a system 10 for creating a biocompatible and biodegradable barrier to seal a vascular puncture site. The system includes a barrier composition comprising a biocompatible, synthetic electrophilic polymer component 12 that is mixed with a biocompatible, synthetic nucleophilic component 14 that includes Poly-L-Lysine hydrobromide. The solid components 12 and 14 are preferably in solution for delivery.

The system 10 also includes a delivery device 16 for delivering the components 12 and 14 of the barrier material to an arteriotomy (vascular puncture) site created by standard Seldinger technique.

1. Electrophilic Component

In the illustrated embodiment, the electrophilic component 12 comprises a synthetic hydrophilic polymer. The hydrophilic polymer comprises a poly(ethylene glycol) (PEG) Succinimidyl Glutarate having a functionality of four—or, in short hand, 4-Arm PEG Succinimidyl Glutarate (PEG-SG)—having a molecular weight of about 10,000 g/mole (available from Polymer Source, Inc. at www.polymersource.com).

The 4-Arm PEG-SG is dissolved in Sterile Water for Injection WFI USP (available from Abbott Laboratories) for delivery. In a representative embodiment, a targeted weight of 0.25 g of 4-Arm PEG-SG is mixed in a targeted volume of 1.25 cc of Sterile Water for Injection (WFI) USP and mixed. No buffering material is added. One (1) cc of the resulting WFI/PEG-SG solution is housed in a sterile dispensing syringe 18. The delivery device 16 receives the dispensing syringe 18 during use, as will be described in greater detail later.

2. The Nucleophilic Component

In the illustrated embodiment, the nucleophilic component 14 includes a blend of a poly(ethylene glycol) (PEG) Amine having a functionality of four—or, in short hand, 4-Arm PEG Amine—having a molecular weight of about 10,000 g/mole (available from Polymer Source, Inc. at www.polymersource.com), and a Poly-L-Lysine hydrobromide (HBr) having a molecular weight of greater than about 8000 g/mole (available from Albumin Therapeutics, Inc. or ICN Biomedicals, Inc. at www.mpbio.com). Poly-L-Lysine hydrobromide is not characterized in terms of “functionality” as are PEG materials (i.e., 4-Arm PEG means a PEG with a functionality of four). Poly-L-Lysine hydrobromide is a polypeptide moiety (like albumin) that is characterized not by “functionality” but by reference to the number of active surface lysines, which for Poly-L-Lysine hydrobromide is at least twenty (20) per 5000 M/W.

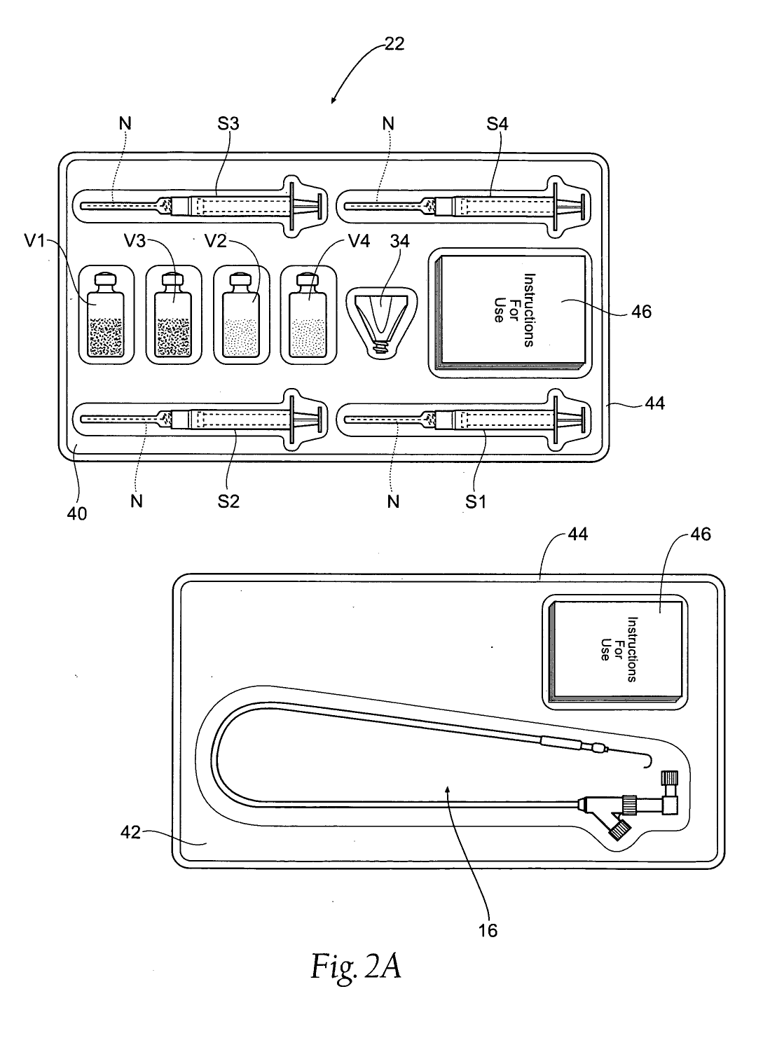

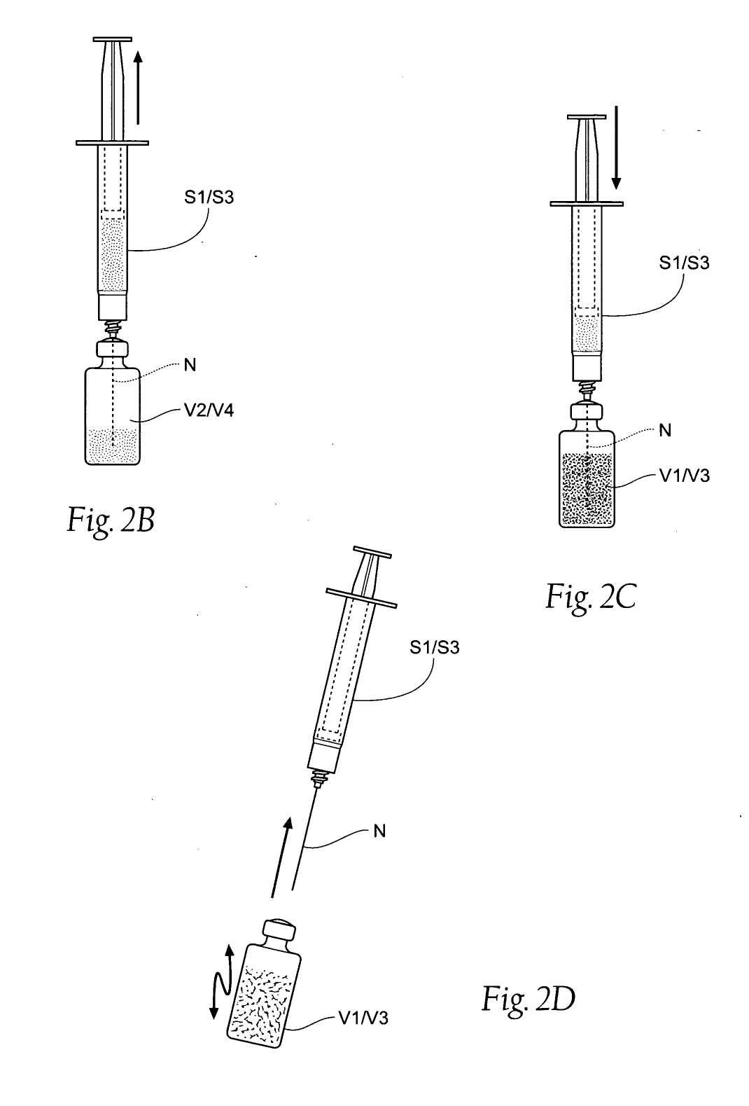

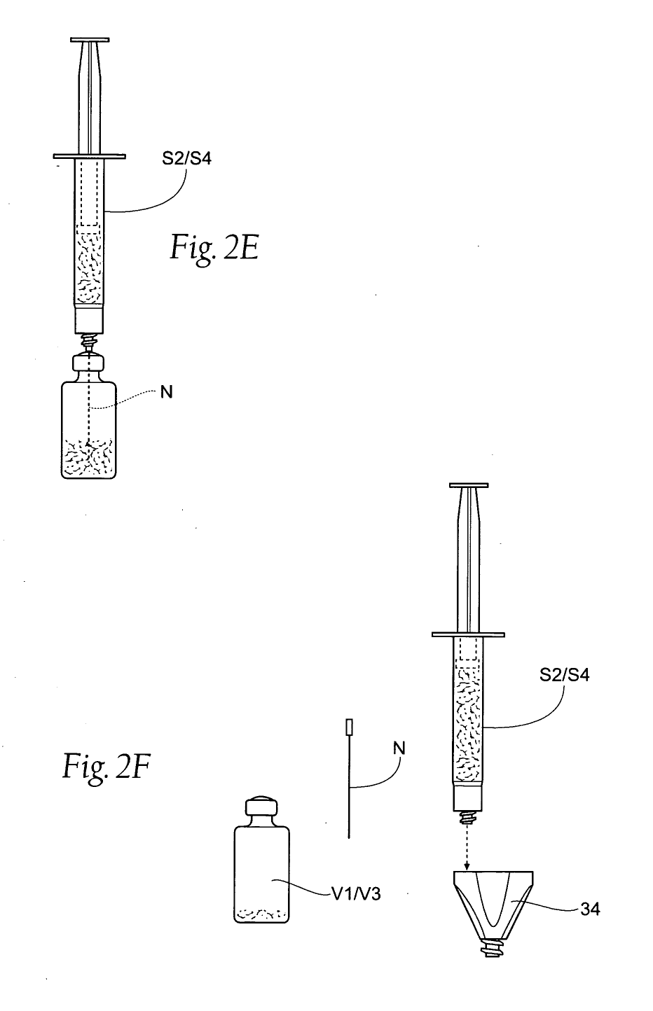

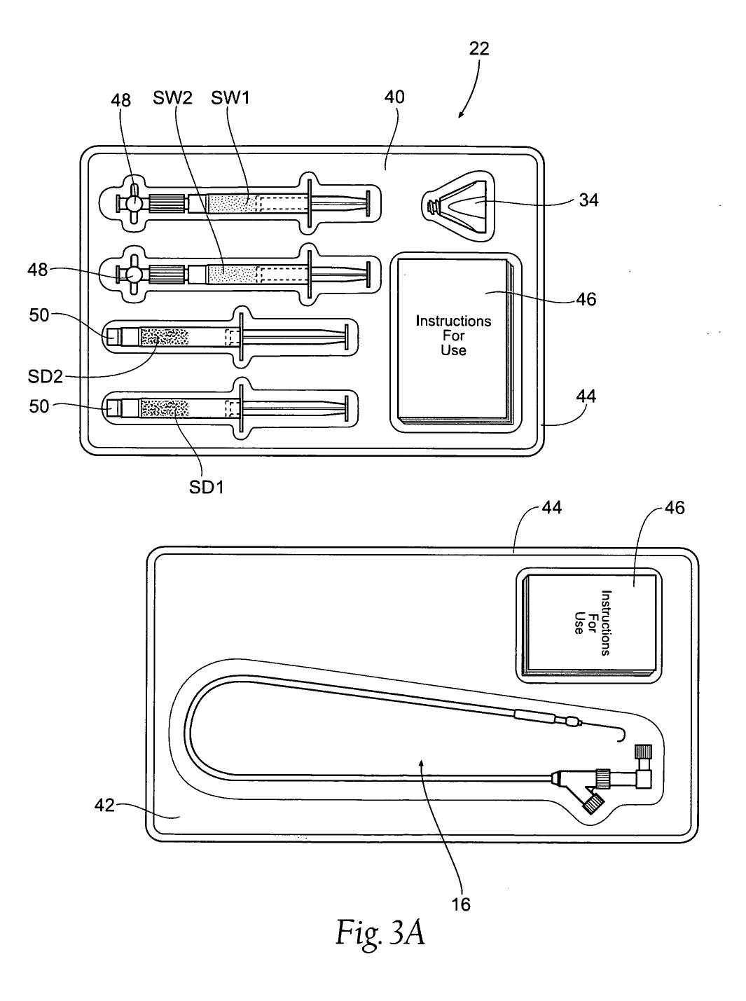

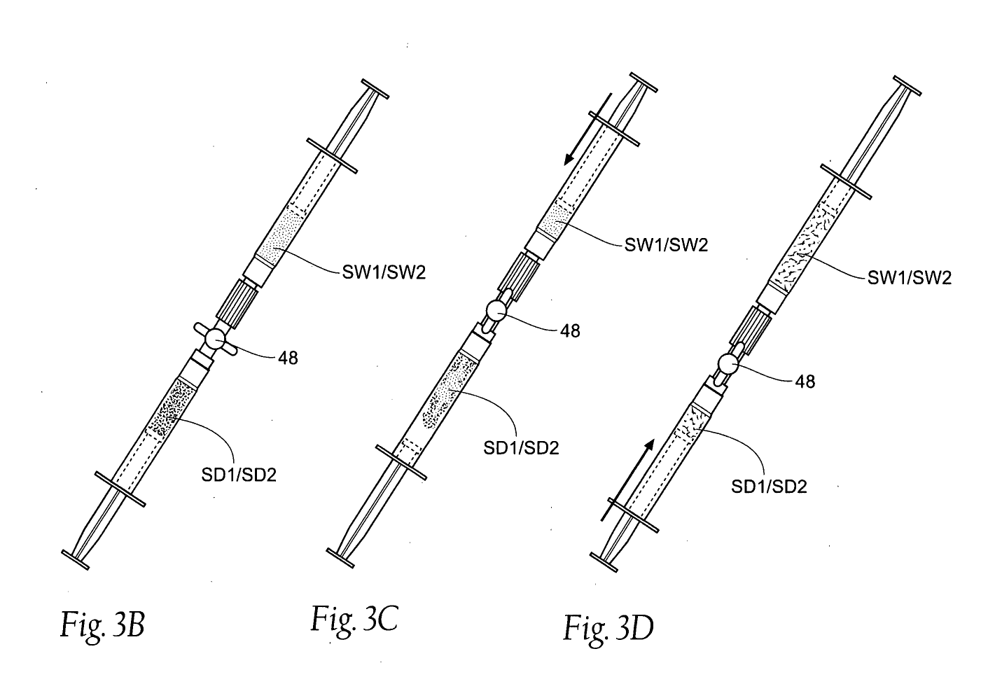

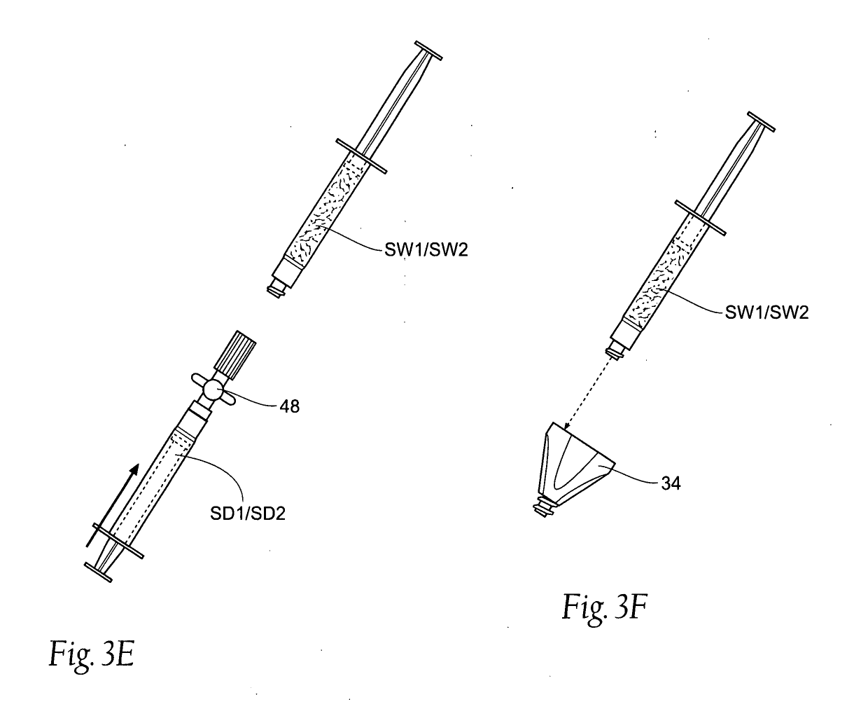

The 4-Arm PEG-Amine and Poly-L-Lysine hydrobromide are dissolved in HPLC-grade water for delivery. In a representative embodiment, a targeted weight of 0.14 g of PEG-Amine and a target weight of 0.039 g of the Poly-L-Lysine hydrobromide are added to a target volume of 1.25 cc of HPLC-grade water to which a buffer material, such as tris(hydroxymethyl)aminomethane buffer, is added to achieve a pH between about 9.0 and 9.9, preferably between about 9.25 to 9.8, and most preferably at about 9.6. The buffered HPLC-grade water, 4-Arm PEG-Amine, and Poly-L-Lysine hydrobromide are mixed in solution. One (1) cc of the HPLC Water/PEG-Amine/Poly-L-Lysine hydrobromide solution is housed in a second sterile dispensing syringe 20. The delivery device 16 receives the second dispensing syringe 20 along with the first described syringe 18 during use, as will be described in greater detail later. As shown in FIGS. 2A and 3A, and as will be described in greater detail later, kits 22 may be provided to facilitate mixing of the electrophilic and nucleophilic components 12 and 14 on site at the instant of use.

In the illustrated compositions, it has been discovered that the maintenance of two different weight-to-weight ratios is important.

The first ratio is the weight-to-weight ratio between the 4-Arm PEG-Amine in the second component 14 and the 4-Arm PEG-SG in the first component 12. This weight-to-weight ratio is calculated by dividing the weight (in g) of 4-Arm PEG-Amine in the composition by the weight (in g) of the 4-Arm PEG-SG in the composition. This weight-to-weight ratio is selected to be about 0.5 to less than 1.0. This weight-to-weight ratio assures that there will be a greater amount of 4-Arm PEG-SG functional groups than 4-Arm PEG-Amine functional groups. This selected ratio provides that substantially all 4-Arm PEG-Amine functional groups will be reacted with the 4-Arm PEG-SG functional groups during the cross-linking process. The substantial absence of unreacted amine functional groups enhances the overall biocompatibility of the resulting hydrogel.

The second ratio is the weight-to-weight ratio between the Poly-L-Lysine hydrobromide and the 4-Arm PEG-Amine. This weight-to-weight ratio is calculated by dividing the weight (in g) of Poly-L-Lysine hydrobromide in the composition by the weight (in g) of the 4-Arm PEG-Amine in the composition. This weight-to-weight ratio is selected to be less than about 0.5, and preferably about 0.2 to 0.3, i.e., there is significantly less Poly-L-Lysine hydrobromide than 4-Arm PEG-Amine. It has been discovered that this weight-to-weight ratio provides a delay in the gelation process of the hydrogel for a period of time after mixing the two components 12 and 14, during which viscosity of the mixture does not appreciably change. The delay in gelation, which will also in shorthand be called the “open time,” is beneficial. The open time allows for passage of the two components 12 and 14 through the delivery device 16 without gelation. Passage of the components 12 and 14 can therefore occur without clogging the delivery device 16. Gelation occurs after the components 12 and 14 exit the catheter shaft 22, at the vascular puncture site. Maintaining this selected weight-to-weight ratio between the Poly-L-Lysine hydrobromide and the 4-Arm PEG-Amine also assures that the resulting hydrogel is flexible and not brittle. As will be demonstrated, a composition comprising 4-Arm PEG-Amine and 4-Arm PEG-SG (with no Poly-L-Lysine hydrobromide component) does not provide the open time. As will also be demonstrated, a composition comprising Poly-L-Lysine hydrobromide and a 4-Arm PEG-SG (with no 4-Arm PEG-Amine component) also does not provide the open time.

3. The Delivery Device

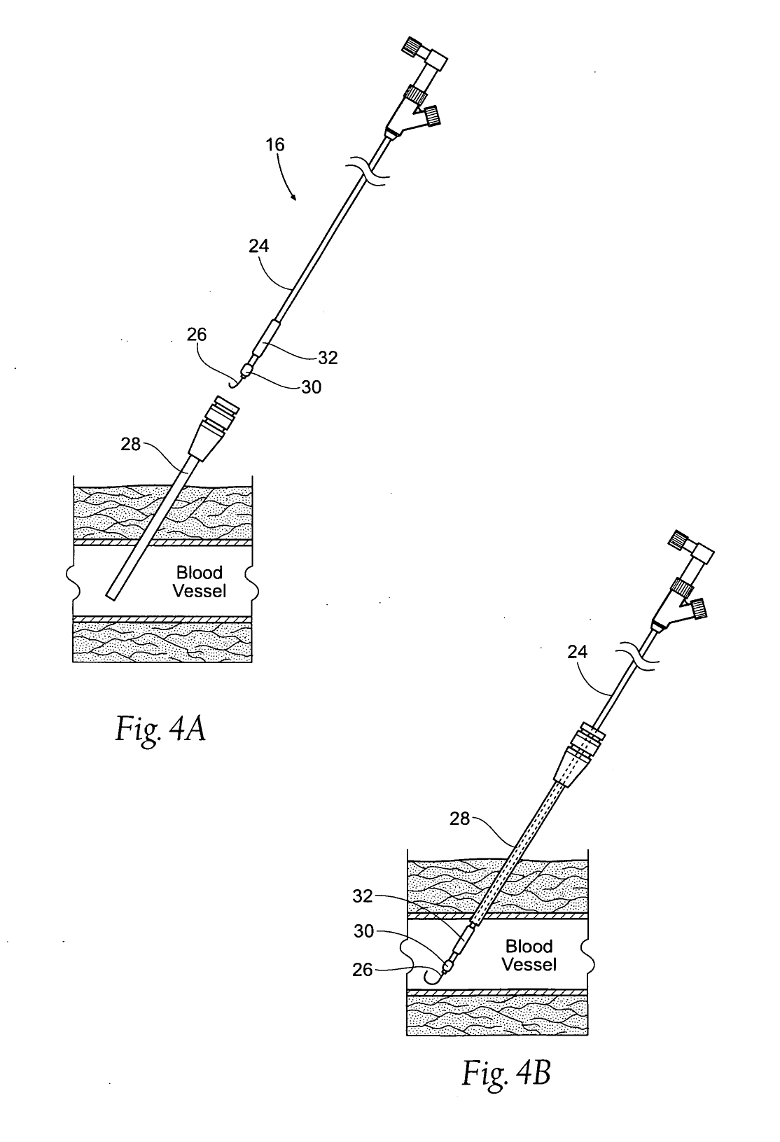

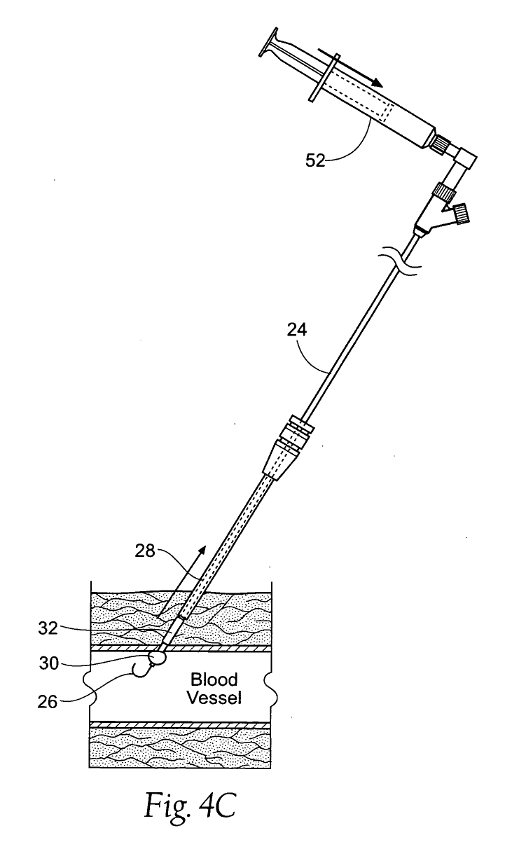

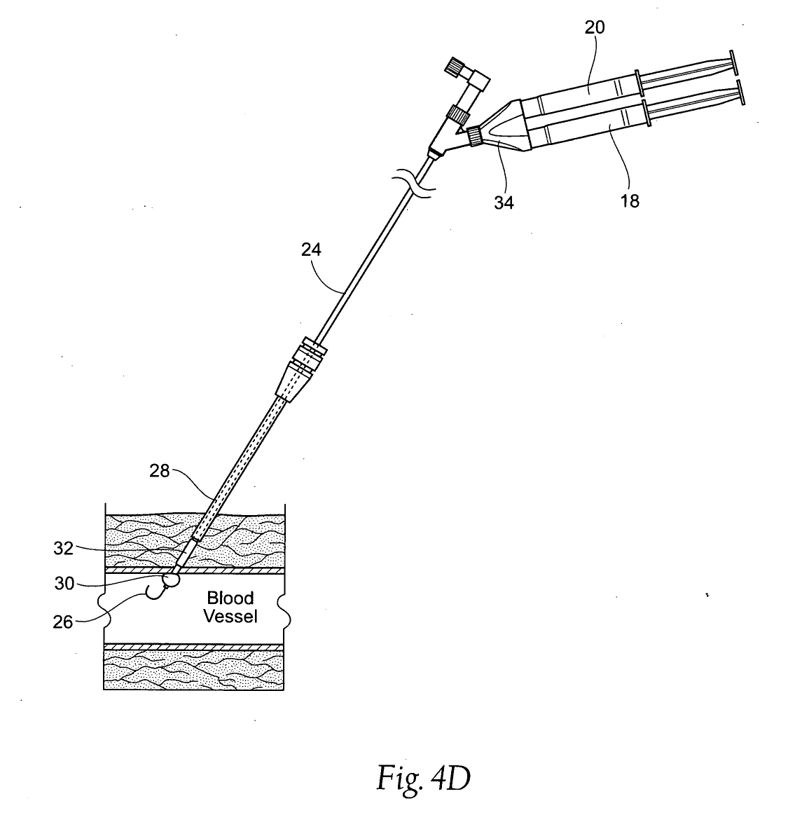

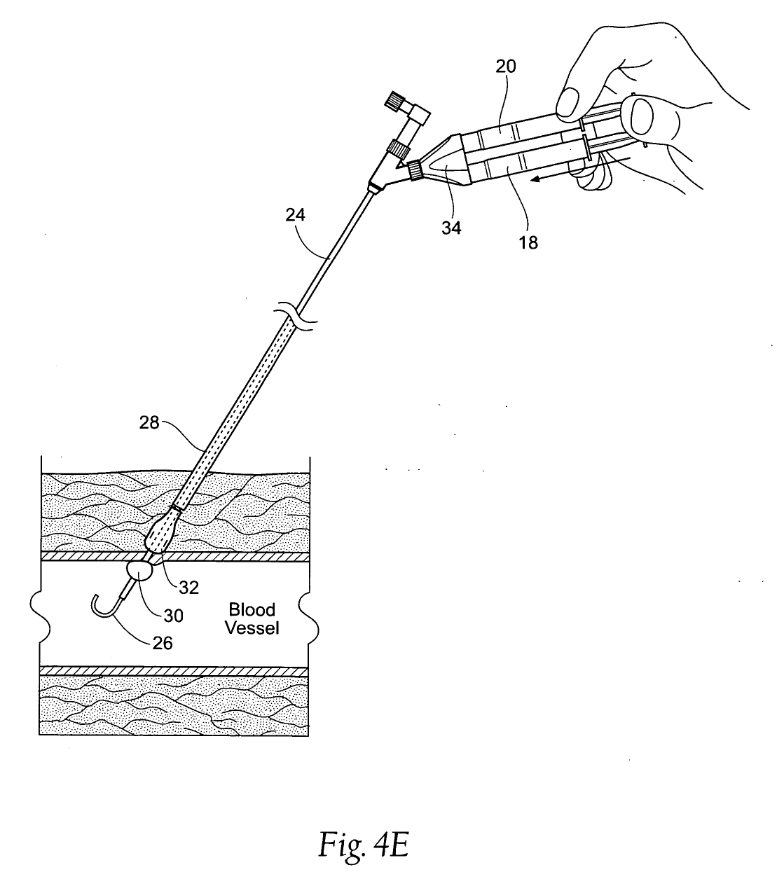

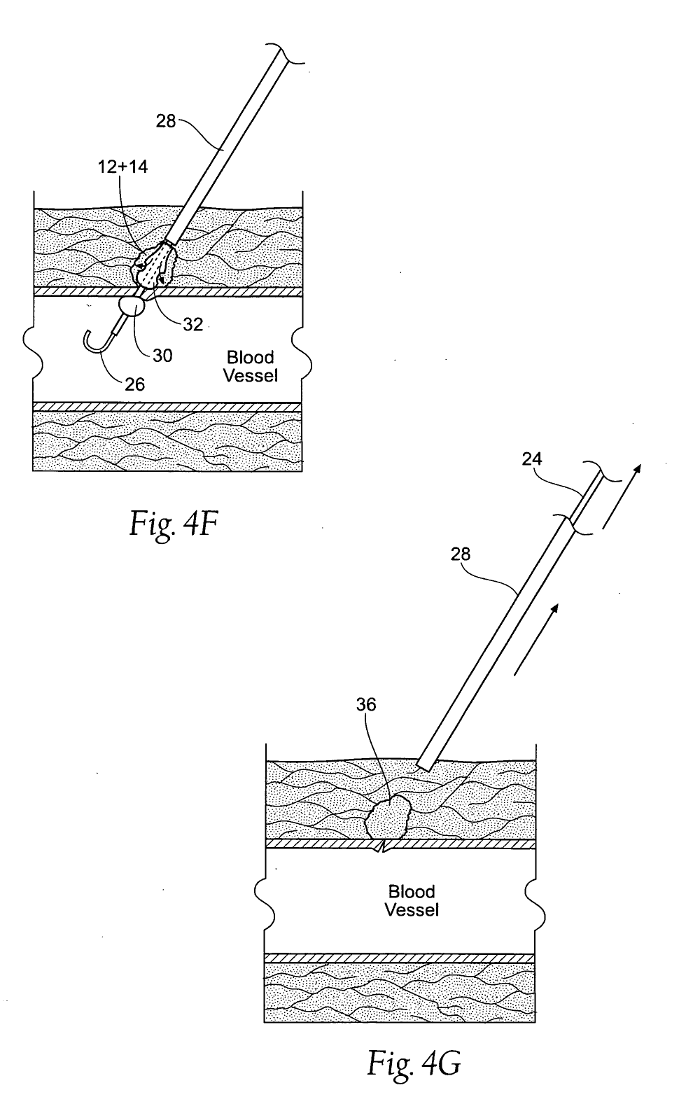

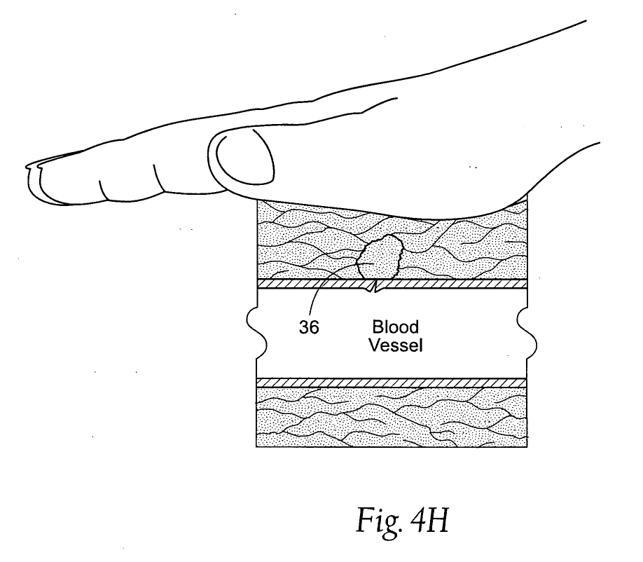

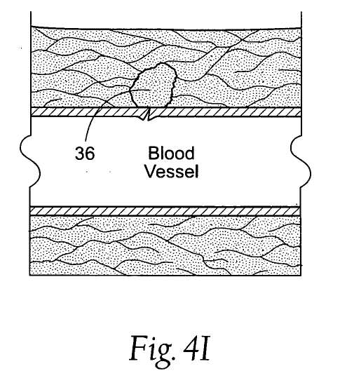

The delivery device 16 can be variously constructed. As shown in FIG. 1, the delivery device 16 comprises a MedClose™ Extravascular Delivery Syringe of the type shown, e.g., in U.S. Pat. No. 5,725,551, which is incorporated herein by reference. The delivery device 16 includes a 5 Fr catheter shaft 24 (20 cm long) with fixed J-tip guidewire 26. As further shown in FIGS. 4A and 4B, the catheter shaft 24 is sized and configured for insertion into a conventional introducer sheath 28 (e.g., 6 Fr. to 9 Fr. or larger) that has been placed into blood vessel, e.g., a femoral artery, using a conventional Seldinger technique.

As FIG. 4C further shows, the catheter shaft 22 includes a small, compliant balloon 30 at its distal tip. After passage through the sheath 28 into the vessel lumen, the distal balloon 30 is inflated, e.g., using an inflation syringe 54, as shown in FIG. 4C. The catheter shaft 24 and sheath 28 are proximally withdrawn to place the inflated balloon 30 against the vessel wall to create a temporary barrier to bleeding, while also preventing intravascular passage of material from outside the vessel. This is also shown in FIG. 4C.

As FIGS. 4D and 4E further show, the catheter shaft 24 also includes a second, more proximal, perforated elastic delivery sleeve 32. The sleeve 32 is sized and configured to occupy the needle tract beyond the end of the sheath 28. The components 12 and 14 are dispensed from the delivery syringes 18 and 20 through a Y-Joiner 34 coupled to the proximal end of the catheter shaft 24. The components 12 and 14 flow through a lumen in the delivery shaft 24 into the perforated delivery sleeve 32, mixing in the process. As FIG. 4F further shows, the components 12 and 14, now mixed, are “squeezed” out of the perforated, elastic delivery sleeve 32 and into the tissue tract proximal to the vessel wall. After, e.g., about 45 to 60 seconds after delivery of the components 12 and 14, the distal balloon 30 can be deflated, and both the catheter shaft 24 and introducer sheath 28 can be simultaneously withdrawn from the puncture site, as FIG. 4G shows. Manual compression is desirably maintained on the puncture site for a subsequent period of time, e.g., 2 to 3 minutes (as FIG. 4H shows).

The two components 12 and 14 mixed in a liquid state during passage through the catheter shaft 24, become reactive after they are delivered to the vascular puncture site outside the vessel wall. When mixed, the two components 12 and 14 begin to react by cross-linking, forming a solid matrix composition 36, or hydrogel, as FIG. 4I shows.

As will be demonstrated below, due to the composition of the first and second components 12 and 14, and in particular the selected weight-to-weight ratio between Poly-L-Lysine hydrobromide and the 4-Arm PEG-Amine, the two components 12 and 14 do not immediately react, but exhibit an “open time” after mixing before gelation. The “open time” allows passage of the components 12 and 14 through the catheter shaft 24 to mix, but not gel. The gelation occurs at the puncture site outside the catheter shaft 22. The formed hydrogel 36 possesses high gel strength, adhesive properties, and cohesive properties to bring about hemostasis in situ within about three to five minutes.

4. The Kits

As FIGS. 2A and 3A show, the components of the system can be individually provided for use in sterile kits 22. In the illustrated embodiment, each kit 22 includes an interior tray 40/42 made, e.g., from die cut cardboard, plastic sheet, or thermo-formed plastic material. The delivery device 16 is carried by a tray 42 in one kit 22. The first and second components 12 and 14 of the system 10 are carried by a tray 40 in another kit 22. The solid and liquid constituents of the first and second components 12 and 14 are individually packaged in the tray 40, as are the devices (e.g., syringes) needed to mix the solid and liquid constituents of the first and second components 12 and 14 together at the instance of use. Also carried in the second kit is the Y-Joiner 34 that, in use, is coupled to the catheter device 22.

Each tray includes a tear-away overwrap 44, to peripherally seal each tray 40 and 42 from contact with the outside environment. The kit 22 carrying delivery device 16 can be sterilized by convention ethylene oxide (ETO) sterilization techniques. The kit 22 carrying the first and second components 12 and 14 can be sterilized using conventional electron beam (E-Beam) sterilization.

In the illustrated embodiment, each kit 22 also preferably includes directions or instructions 46 for using the contents of the kit to carry out a desired procedure. Exemplary directions will be described later. The directions or instructions 46 can, of course vary, according to the particularities of the desired procedure. Furthermore, the directions or instructions 46 need not be physically present in the kits 22. The directions or instructions 46 can be embodied in separate instruction manuals, or in video or audio tapes, or in electronic form.

The kit 22 shown in FIG. 2A includes four vials V1, V2, V3, and V4, each sealed by a self-closing septum that can be needle-pierced. The vial V1 includes a targeted weight volume of the 4-Arm PEG-SG material in an inert atmosphere (e.g., argon) in a stable, powder form. Likewise, the vial V3 includes targeted weight volumes of the 4-Arm PEG-Amine and the Poly-L-Lysine hydrobromide materials in an inert atmosphere (e.g., argon) in a stable, powder form. The vial V2 includes a volume of Sterile Water for Injection (WFI) USP for mixing with the 4-Arm PEG-SG in the vial V1. The vial V4 includes a volume of buffered HPLC-grade water (pH=9.65) for mixing with the 4-Arm PEG-Amine and the Poly-L-Lysine hydrobromide materials in the vial V3.

The kit 22 shown in FIG. 2A also includes four syringes, S1, S2, S3, and S4, each carrying a needle N that is removably fitted to the syringe S1, S2, S3, and S4 by a threaded luer fitting. The instructions 46 direct the use of the syringes S1, S2, S3, and S4 to mix the first and second components at the instance of use.

To prepare the first component 12, the instructions 46 direct using the first syringe S1 to transfer 1.25 cc of the WFI USP from the vial V2 into the contents of the vial V1, as FIGS. 2B and 2C show. The instructions 46 also direct discarding the first syringe S1 and shaking the vial V1 to mix the 4-Arm PEG-SG with the WFI USP, as FIG. 2D shows. The instructions 46 further direct withdrawing 1 cc of the mixture from vial V1 into the second syringe S2, as FIG. 2E shows. The instructions 46 then direct removal of the needle from the second syringe S2 and joining the second syringe S2 to the Y-Joiner 34 for use, as FIG. 2F shows.

To prepare the second component 14, the instructions 46 likewise direct the same sequence of steps using the syringes S3 and S4 on concert with the vials V3 and V4. The instructions 46 direct using the third syringe S3 to transfer 1.25 cc of the buffered HPLC-grade water from the vial V4 into the contents of the vial V3, as FIGS. 2B and 2C show. The instructions 46 also direct discarding the third syringe S3 and shaking the vial V3 to mix the 4-Arm PEG-Amine and Poly-L-Lysine hydrobromide with the buffered HPLC-grade water, as FIG. 2D shows. The instructions 46 further direct withdrawing 1 cc of the mixture from vial V3 into the fourth syringe S4, as FIG. 2E shows. The instructions 46 then direct removal of the needle from the fourth syringe S4 and joining the fourth syringe S4 to the Y-Joiner 34 for use, as FIG. 2F shows.

The instructions 46 direct coupling the Y-Joiner 34 and syringes (S2 corresponds to syringe 18 and S4 corresponds to syringe 20) to the delivery device 16, as is shown in FIG. 4D.

The kit shown in FIG. 3A includes two dry syringes SD1 and SD2, each sealed by a removable cap 50. The dry syringe SD1 includes a targeted weight volume of the 4-Arm PEG-SG material in an inert atmosphere (e.g., argon) in a stable, powder form. Likewise, the dry syringe SD2 includes targeted weight volumes of the 4-Arm PEG-Amine and the Poly-L-Lysine hydrobromide materials in an inert atmosphere (e.g., argon) in a stable, powder form.

The kit 22 shown in FIG. 3A also includes two wet syringes SW1 and SW2, each sealed with a stopcock valve 48 secured by a threaded luer fitting. The wet syringe SW1 is sized (e.g., at 3 cc) to accommodate a volume (1.25 cc) of Sterile Water for Injection (WFI) USP for mixing with the 4-Arm PEG-SG in the dry syringe SD1. The wet syringe SW2 is also sized (e.g., at 3 cc) to accommodate a volume of buffered HPLC-grade water (pH=9.65) (1.25 cc) for mixing with the 4-Arm PEG-Amine and the Poly-L-Lysine hydrobromide materials in the dry syringe SD2.

The instructions 46 in the kit shown in FIG. 3A direct the use of the dry syringes SD1, SD2 and the wet syringes SW1, SW2 to mix the first and second components 12 and 14 at the instance of use.

To prepare the first component 12, the instructions 46 direct coupling the dispensing end of the dry syringe SD1 to the stopcock valve 48 on the wet syringe SW1, as shown in FIG. 3B. The stopcock valve 48 is closed at this point. The directions then direct opening the stopcock valve 48 (see FIG. 3C) and transferring the WFI USP from the wet syringe SW1 into the dry syringe SD1, as FIG. 3C shows. The instructions 46 direct to repeatedly transfer the water and powder mixture between the two syringes SW1 and SD1, as FIGS. 3C and 3D show, to syringe-to-syringe mix the powder and water until all solids are dissolved. The syringe-to-syringe mixing places the 4-Arm PEG-SG into solution. The instructions 46 then direct withdrawing 1 cc of the mixture from the dry syringe SD1 into the wet syringe SW1 and disconnecting the wet syringe SW1 from the stopcock valve 48, as FIG. 3E shows. The instructions 46 then direct joining the wet syringe SW1 to the Y-Joiner 34 for use, as FIG. 3F shows.

To prepare the second component 14, the instructions 46 likewise direct the same sequence of syringe-mixing steps using the wet syringe SW2 and the dry syringe SD2. The instructions 46 direct coupling the dispensing end of the dry syringe SD2 to the stopcock valve 48 on the wet syringe SW2, as shown in FIG. 3B. The stopcock valve 48 is closed at this point. The directions then direct opening the stopcock valve 48 (see FIG. 3C) and transferring the buffered HPLC-grade water from the wet syringe SW2 into the dry syringe SD2, as FIG. 3C shows. The instructions 46 direct to repeatedly transfer the water and powder mixture between the two syringes SW2 and SD2, as FIGS. 3C and 3D show, to syringe-to-syringe mix the powder and water until all solids are dissolved. The syringe-to-syringe mixing places the 4-Arm PEG-Amine and Poly-L-Lysine hydrobromide into solution. The instructions 46 then direct withdrawing 1 cc of the mixture from the dry syringe SD2 into the wet syringe SW2 and disconnecting the wet syringe SW2 from the stopcock valve 48, as FIG. 3E shows. The instructions 46 then direct joining the wet syringe SW2 to the Y-Joiner 34 for use, as FIG. 3F shows. The instructions 46 direct coupling the Y-Joiner 34 and syringes (SW1 corresponds to syringe 18 and SW2 corresponds to syringe 20) to the delivery device 16, as is shown in FIG. 4D.

The instructions 46 can suggest time intervals for deploying and withdrawing the delivery device 16 in conjunction with the delivery of the components 12 and 14 to form the hydrogel 36. The time intervals take into account the chemical characteristics and reactivity of the components 12 and 14.

For example, the instructions 46 can suggest, in a first sequence, the delivery of the components 12 and 14 from the syringes 18 and 20 through the delivery device 16 during a period of 10 to 15 seconds. This sequence is generally shown in FIG. 4E.

The instructions 46 can suggest, in a next sequence, keeping the delivery device 16 residing within the vessel puncture site for about 45 seconds after the initial delivery period. This sequence is generally shown in FIG. 4F. This pause allows the components 12 and 14 to exit the device 16 and start the cross-linking process (i.e., the gelation process). During this time, the cross linking of the components 12 and 14 achieves a semi-solid state, creating a matrix that is no longer liquid, but does not possess its full gelation properties.

The instructions 46 can suggest, in a next sequence immediately following the pause just described, withdrawing the delivery device 16 and sheath 28 from the vessel puncture site. This is generally shown in FIG. 4G. Since the cross-linking matrix is at this time still semi-solid, the catheter shaft 24 can be withdrawn through the matrix, and the matrix will subsequent continue to cross-link and close in about the withdrawal path.

The instructions can suggest, immediately following the withdrawal of the delivery device 16 and sheath 28, the application of manual pressure on the site of the tissue tract. This is generally shown in FIG. 4H. During this period, e.g., occupying an interval of about 3 minutes following the withdrawal of delivery device 16 and sheath 28, the gel strength, adhesive properties, and cohesive properties of the hydrogel 36 progressively increase to bring about hemostasis in situ.

### Example 1

Preparation of the Electrophilic Component:

A weight of 0.256 g of 4-Arm PEG-SG (M/W 10,000 g/mole) is added to a volume of 1.25 cc of Sterile Water for Injection (WFI) USP, and mixed in one of the manners described above. No buffering material is added. One (1) cc of the resulting WFI/PEG-SG solution is housed in a sterile dispensing syringe, as described.

Preparation of the Nucleophilic Component:

A weight of 0.134 g of 4-Arm PEG-Amine (M/W 10,000 g/mole) and a weight of 0.033 g of the Poly-L-Lysine hydrobromide (M/W greater than about 8000 g/mole) are added to a volume of 1.25 cc of HPLC-grade water (buffered to a pH 9.724, e.g., with tris(hydroxymethyl)aminomethane buffer material), and mixed in one of the manners previously described. One (1) cc of the buffered HPLC Water/4-Arm PEG-Amine/Poly-L-Lysine hydrobromide solution is housed in a sterile dispensing syringe, as described.

Mixing of the Components/Formation of the Hydrogel:

A volume of 1 cc of the prepared electrophilic component 12 is mixed with a volume of 1 cc of the prepared nucleophilic component 14 (total mixed volume=2 cc) in the manner previously described.

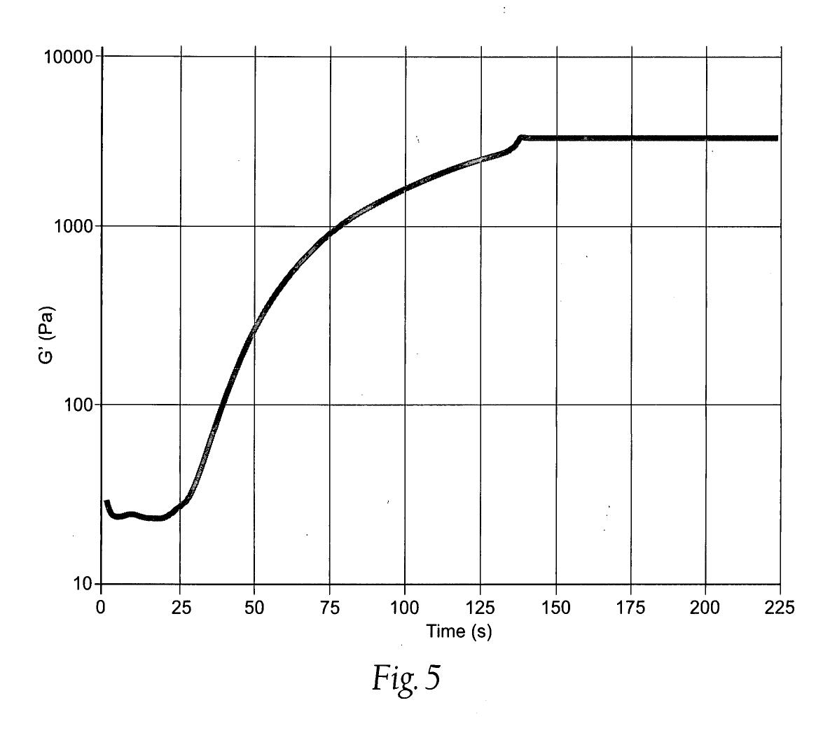

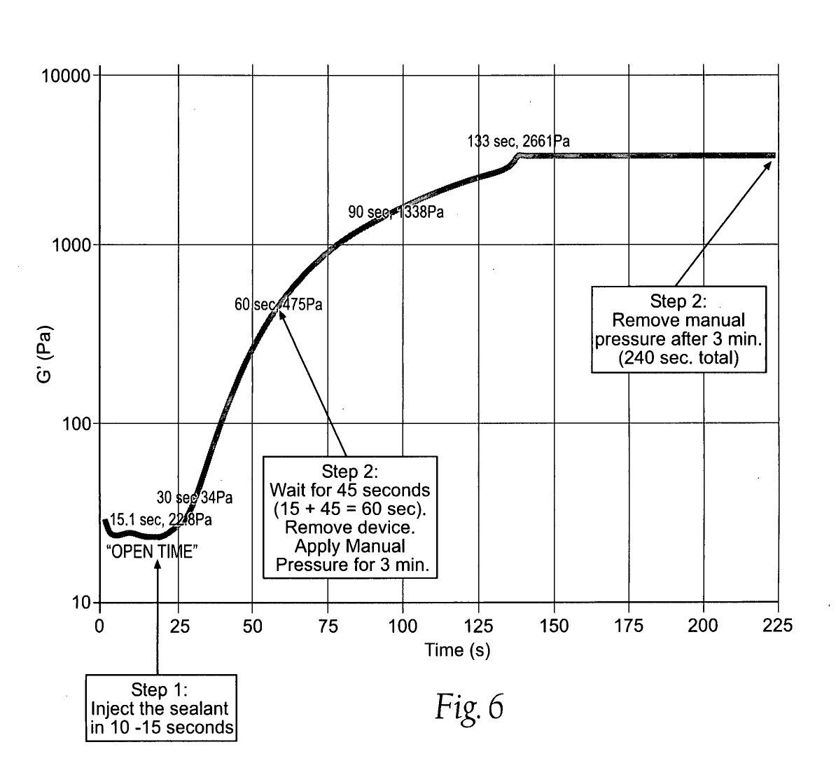

The accumulating gel strength G′ (in Pascals or Pa) of the mixture over time is measured on a TA Instruments (New Castle, Del.) Model No. AR2000EX Rheometer: 2% strain, in oscillation mode frequency 1 Hz fast oscillation mode, 10 data points per second, time sweep, 25 mm plate, 1.5 mm gap, at 25-degrees C. The resulting graph of G′ (in Pascels) over time is shown in FIG. 5. FIG. 6 shows the same graph, but also interlays gelation strengths at various time intervals and relates these intervals to the sequence of delivery just described and as also shown in FIGS. 4D to 4I.

FIGS. 5 and 6 demonstrate that the described composition exhibits a delay in gelation for about 25 seconds after mixing, which is called “open time.” During this open time, viscosity does not change. The “open time” is beneficial, because it allows for passage of the two components 12 and 14 through the lumen of the delivery device 16 without gelation. Passage of the components 12 and 14 can therefore occur without clogging the lumen of the delivery device 16. Gelation occurs later, after the components 12 and 14 have exited the delivery device 16 and reside proximal to the vascular puncture site.

FIGS. 5 and 6 also show rapidly accumulating gel strengths after the open time: of 34 G′ (Pascals) at 30 seconds after mixing; 475 G′ (Pascals) at 60 seconds after mixing; 1338 G′ (Pascals) at 90 seconds after mixing; and 2661 G′ (Pascals) at 133 seconds after mixing.

At about 60 seconds after delivery (delivery interval of 10 to 15 seconds, followed by a pause of 45 seconds), the gel strength of 475 G′ (Pascals) is about twenty percent (20%) of final gel strength of 2661 G′ (Pascals). This semi-solid, not fully gelled state permits withdrawal of the delivery device 16 and sheath 28 from the vessel puncture site without compromising the integrity of the forming hydrogel. The hydrogel will continue to cross-link and close in (self-seal) about the withdrawal path.

During the next three minutes, application of manual pressure as described compliments the progressive increase in gel strength up to 2661 G′ (Pascals).

The result is a very strong gel, as the “chattering” observed in the graph in FIGS. 5 and 6 after 133 seconds demonstrates. The gel demonstrates excellent adhesive properties and cohesive properties in a time period well suited for vascular puncture sealing (in a delivery and gelation period of about four total minutes, i.e., 240 seconds).

### Example 2

Aliquot volumes of 1 cc each of the electrophilic component 12 (4-Arm PEG-SG (M/W 10,000 g/mole,) and aliquot volumes of 1 cc each of the nucleophilic component 14 (4-Arm PEG-Amine (M/W 10,000 g/mole and Poly-L-Lysine hydrobromide (M/W greater than about 8000 g/mole) were prepared in the weight amounts shown in the following table:

| | Nucleophilic | 1 cc Total Blend | Electrophilic | |
|---|---|---|---|---|
| | Component | Volume | Component | |
| | Blend | 4-Arm PEG-Amine (M/W | (1 cc Volume) | |
| | Poly-L- | 10,000 g/mole) | 4-Arm PEG-SG | |
| | Lysine | in 1.25 cc of HPLC- | (M/W 10,000 g/ | |
| | hydrobromide | grade water, pH 9.65, | mole) in | |
| | (M/W Greater | buffered with, with | 1.25 cc | |
| Formulation | Than About | tris (hydroxymethyl) | sterile water | |
| Number | 8000 g/mole) | aminomethane | (WFI) | Comments |
| 1 | 0.0405 g | 0.1400 g | 0.2506 g | Successful |
| | | | | Hemostasis |
| | | | | 6 Fr |
| | | | | Introducer |
| | | | | Sheath |
| 2 | 0.0409 g | 0.1447 g | 0.2511 g | Successful |
| | | | | Hemostasis |
| | | | | 7 Fr |
| | | | | Introducer |
| | | | | Sheath |
| 3 | 0.0401 g | 0.1444 g | 0.2500 g | Successful |
| | | | | Hemostasis |
| | | | | 8 Fr |
| | | | | Introducer |
| | | | | Sheath |
| 4 | 0.0407 g | 0.1425 g | 0.2522 g | Successful |
| | | | | Hemostasis |
| | | | | 9 Fr |
| | | | | Introducer |
| | | | | Sheath |

The Formulations 1, 2, 3, and 4 were sterilized by E-Beam sterilization to 30 kGy.

Using a MedClose™ Extravascular Delivery Syringe and the mixing and delivery techniques described above, Formulations 1, 2, 3, and 4 were delivered individually through, respectively, 6 Fr; 7 Fr; 8 Fr; and 9 Fr introducer sheaths. The sheaths had been inserted using standard Seldinger technique in a contralateral femoral artery of a normal, anesthesized 150 lb pig. The pig had been heparinized for Active Clotting Time above 150.

In each formulation, 1 cc of the 4-Arm PEG-SG (M/W 10,000 g/mole) was mixed during delivery with 1 cc of the blend of the Poly-L-Lysine hydrobromide (M/W greater than about 8000 g/mole) and 4-Arm PEG-Amine (M/W 10,000 g/mole). For each delivery, there was a two minute period for deployment of the sheath 28 and delivery device 16 (including a 10 to 15 second injection period), a wait period for forty-five (45) seconds prior to withdrawal of the delivery device and sheath; and a manual compression of the puncture side for three (3) subsequent minutes.

The delivery of all four Formulations 1, 2, and 3 through their respective sheaths achieved successful hemostasis, as defined as the cessation of bleeding from the arterial puncture site wound within 5 minutes following percutaneous injection of the sealant in the introducer tract, followed by manual compression.

Successful hemostasis for Formulation 4 (using a 9 Fr Introducer Sheath) was achieved in six total minutes.

No hematoma was evident.

### Example 3

Preparation of the Electrophilic Component:

A weight of 0.25 g of 4-Arm PEG-SG (M/W 10,000 g/mole) is added to a volume of 1.25 cc of Sterile Water for Injection (WFI) USP, and mixed in one of the manners described above. No buffering material is added. One (1) cc of the resulting WFI/PEG-SG solution is housed in a sterile dispensing syringe, as described.

Preparation of the Nucleophilic Component:

A weight of 0.255 g of 4-Arm PEG-Amine (M/W 10,000 g/mole) is added to a volume of 1.25 cc of HPLC-grade water (buffered to a pH 9.177, e.g., with tris(hydroxymethyl)aminomethane buffer material), and mixed in one of the manners previously described. One (1) cc of the buffered HPLC Water/4-Arm PEG-Amine solution is housed in a sterile dispensing syringe, as described.

Mixing of the Components/Formation of the Hydrogel:

A volume of 1 cc of the prepared electrophilic component 12 is mixed with a volume of 1 cc of the prepared nucleophilic component 14 (total mixed volume=2 cc) in the manner previously described.

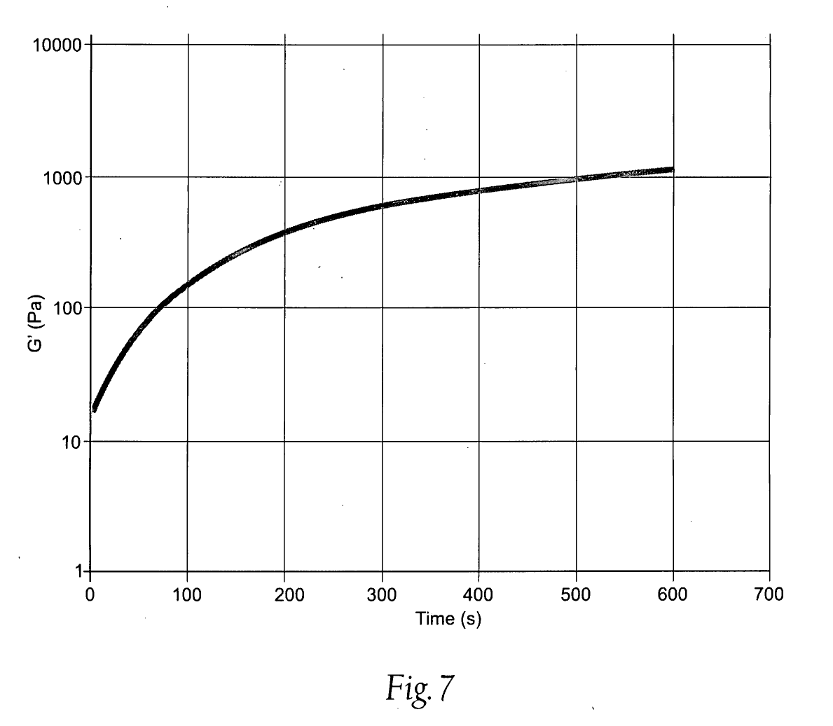

The accumulating gel strength G′ (in Pascals or Pa) of the mixture over time is measured on a TA Instruments (New Castle, Del.) Model No. AR2000EX Rheometer: 2% strain, in oscillation mode frequency 1 Hz fast oscillation mode, 10 data points per second, time sweep, 25 mm plate, 1.5 mm gap, at 25-degrees C. The resulting graph of G′ (in Pascels) over time is shown in FIG. 7.

FIG. 7 shows the absence of the open time shown in FIGS. 5 and 6.

### Example 4

Preparation of the Electrophilic Component:

A weight of 0.25 g of 4-Arm PEG-SG (M/W 10,000 g/mole) is added to a volume of 1.25 cc of Sterile Water for Injection (WFI) USP, and mixed in one of the manners described above. No buffering material is added. One (1) cc of the resulting WFI/PEG-SG solution is housed in a sterile dispensing syringe, as described.

Preparation of the Nucleophilic Component:

A weight of 0.200 g of the Poly-L-Lysine hydrobromide (M/W greater than about 8000 g/mole) is added to a volume of 1.25 cc of HPLC-grade water (buffered to a pH 9.724, e.g., with tris(hydroxymethyl)aminomethane buffer material), and mixed in one of the manners previously described. One (1) cc of the buffered HPLC Water/Poly-L-Lysine hydrobromide solution is housed in a sterile dispensing syringe, as described.

Mixing of the Components/Formation of the Hydrogel:

A volume of 1 cc of the prepared electrophilic component 12 is mixed with a volume of 1 cc of the prepared nucleophilic component 14 (total mixed volume=2 cc) in the manner previously described.

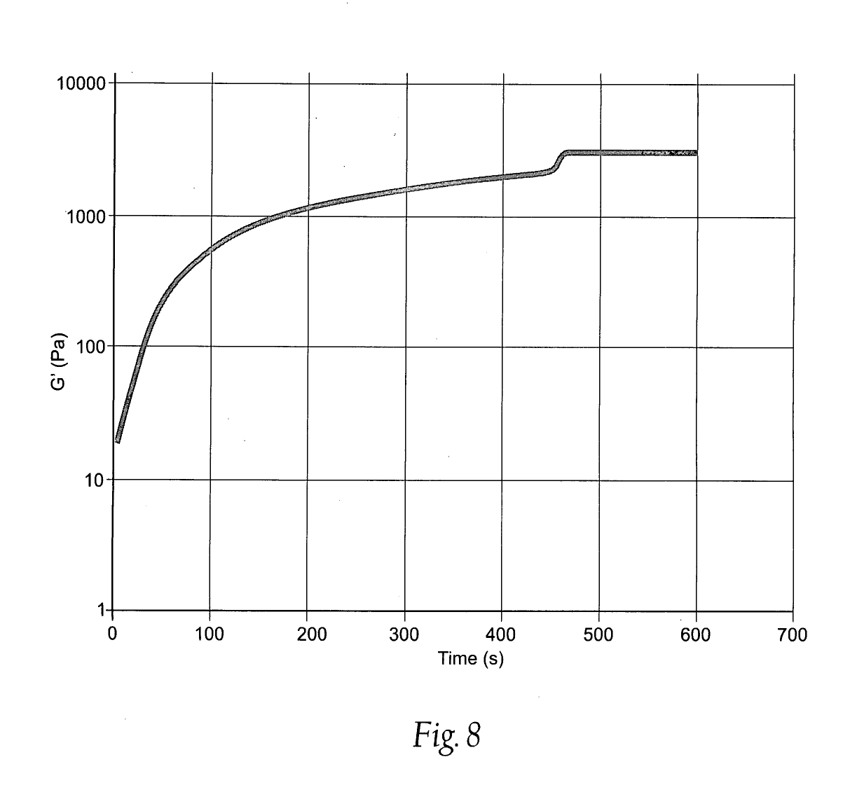

The accumulating gel strength G′ (in Pascals or Pa) of the mixture over time is measured on a TA Instruments (New Castle, Del.) Model No. AR2000EX Rheometer: 2% strain, in oscillation mode frequency 1 Hz fast oscillation mode, 10 data points per second, time sweep, 25 mm plate, 1.5 mm gap, at 25-degrees C. The resulting graph of G′ (in Pascels) over time is shown in FIG. 8.

FIG. 8 shows the absence of the open time shown in FIGS. 5 and 6.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

We claim:

1. A hydrogel composition for application to vascular puncture site of an animal to arrest bleeding and promote hemostasis comprising
a biocompatible, synthetic, electrophilic polymer component comprises a poly(ethylene glycol) (PEG) Succinimidyl Glutarate having a functionality of four and a molecular weight of about 10,000 g/mole, and a biocompatible, synthetic, nucleophilic polymer component comprises a blend of a poly(ethylene glycol) (PEG) Amine having a functionality of four and a molecular weight of about 10,000 g/mole, and a Poly-L-Lysine hydrobromide having a molecular weight of greater than about 8000 g/mole, wherein the weight-to-weight ratio of poly(ethylene glycol) (PEG) Amine to poly(ethylene glycol) (PEG) Succinimidyl Glutarate is about 0.5 to less than 1.0 and the weight-to-weight ratio of Poly-L-Lysine hydrobromide to poly(ethylene glycol) (PEG) Amine is less than about 0.5.

2. A composition according to claim 1
wherein the electrophilic polymer component comprises a targeted weight of 0.25 g of 4-Arm PEG-SG mixed in a targeted volume of 1.25 cc of sterile water free of a buffer material, and delivered in an aliquot of one (1) cc.

3. A composition according to claim 1
wherein the nucleophilic component comprises a targeted weight of 0.14 g of 4-Arm PEG-Amine and a target weight of 0.039 g of Poly-L-Lysine hydrobromide added to a target volume of 1.25 cc of sterile buffered water, and delivered in an aliquot of one (1) cc.

4. A system for applying a biocompatible, non-liquid barrier matrix to seal a vascular puncture site comprising
a catheter device movable into association with the vascular puncture site,
a first dispenser containing a biocompatible, synthetic, electrophilic polymer component comprises a poly(ethylene glycol) (PEG) Succinimidyl Glutarate having a functionality of four and a molecular weight of about 10,000 g/mole,
a second dispenser containing a biocompatible, synthetic, nucleophilic polymer component comprises a blend of a poly(ethylene glycol) (PEG) Amine having a functionality of four and a molecular weight of about 10,000 g/mole, and a Poly-L-Lysine hydrobromide having a molecular weight of greater than about 8000 g/mole, wherein the weight-to-weight ratio of poly(ethylene glycol) (PEG) Amine to poly(ethylene glycol) (PEG) Succinimidyl Glutarate is about 0.5 to less than 1.0 and the weight-to-weight ratio of Poly-L-Lysine hydrobromide to poly(ethylene glycol) (PEG) Amine is less than about 0.5, and
a holder to mutually support the first and second dispensers while conveying the electrophilic polymer component and the nucleophilic polymer component from the dispensers through the catheter device for mixing as a result of flow through the delivery device, wherein, upon mixing, the electrophilic polymer component and the nucleophilic polymer component form the non-liquid barrier matrix at the vascular puncture site.

5. A method of achieving hemostasis at a vascular puncture site comprising
providing a hydrogel composition as defined in claim 1, and
applying the hydrogel composition to a tissue region of the animal.

6. A method comprising
providing a biocompatible, synthetic, electrophilic polymer component comprises a poly(ethylene glycol) (PEG) Succinimidyl Glutarate having a functionality of four and a molecular weight of about 10,000 g/mole,
providing a biocompatible, synthetic, nucleophilic polymer component comprises a poly(ethylene glycol) (PEG) Amine having a functionality of four and a molecular weight of about 10,000 g/mole that upon mixing with the electrophilic polymer component undergoes a gelation process to form a hydrogel, and
delaying onset of the gelation process by blending with the nucleophilic polymer component a Poly-L-Lysine hydrobromide having a molecular weight of greater than about 8000 g/mole at a weight-to-weight ratio of Poly-L-Lysine hydrobromide to poly(ethylene glycol) (PEG) Amine of less than about 0.5.

7. A method according to claim 6
wherein the poly(ethylene glycol) (PEG) Amine is present in the nucleophilic component at a weight-to-weight ratio with the poly(ethylene glycol) (PEG) Succinimidyl Glutarate in the electrophilic component of about 0.5 to less than 1.0.
